(19) United States
(12) Patent Application Publication
(10) Pub. No.: US 2024/0175098 A1
(43) Pub. Date: May 30, 2024

(54) VIRAL CLEARANCE TEST METHOD

(71) Applicant: ASAHI KASEI MEDICAL CO., LTD., Tokyo (JP)

(72) Inventors: Hironobu SHIRATAKI, Tokyo (JP); Juergen EBNER, Vienna (AT)

(73) Assignee: ASAHI KASEI MEDICAL CO., LTD., Tokyo (JP)

(21) Appl. No.: 18/283,596

(22) PCT Filed: Mar. 25, 2022
(86) PCT No.: PCT/JP2022/014401

§ 371 Date: Sep. 22, 2023

(30) Foreign Application Priority Data
Mar. 26, 2021 (JP) 2021-053527

(51) Int. Cl. C12Q 1/70; B01D 69/02; G01N 21/33; G01N 27/06

(52) U.S. Cl. CPC C12Q 1/70; B01D 69/02; G01N 21/33; G01N 27/06

(57) ABSTRACT

The purpose of the present invention is to provide a viral clearance test method, including supplying a protein solution to a first channel provided with, upstream and downstream thereof, a protein purification unit and a virus removal filter, respectively, and pouring the protein solution into the protein purification unit at a first constant rate; supplying, at a second constant rate, a virus solution to a second channel connected between the protein purification unit and the virus removal filter in the first channel and mixing the purified protein solution with the virus solution in the first channel; pouring the mixture of the protein solution and the virus solution into the virus removal filter at a third constant rate; and measuring a virus contained in the permeate of the mixture which has passed through the virus removal filter.

### TECHNICAL FIELD

The present invention relates to a viral clearance test method.

### BACKGROUND ART

Biological material-containing therapeutic drug products include plasma fractionated therapeutic drug products obtained by purifying the blood and biotherapeutic drug products produced by biotechnology. These biological material-containing therapeutic drug products may have a risk of containing a virus. In general, biological material-containing therapeutic drug products should be ensured to be safe against a virus. The production of a biological material-containing therapeutic drug product, therefore, is required to have a step of sufficiently removing or inactivating a virus which is or may be contained in the biological material-containing therapeutic drug product, that is, a virus removal step (refer to, for example, Non-Patent Document 1). A test performed in the virus removal step to determine the virus removal or inactivation capacity is called “viral clearance test”.

What is called “the most robust virus removal step” among the steps of producing a biological material-containing therapeutic drug product is a low pH treatment step or a treatment step with a virus removal medium (refer to, for example, Non-Patent Document 2). The treatment step with a virus removal medium such as virus removal membrane is superior because it can be applied to all the viruses whether they are enveloped or not.

In a viral clearance test in the production steps of a biological material-containing therapeutic drug product, a virus concentration is typically measured before and after an arbitrary production step and a viral clearance capacity is evaluated, for example, by calculating a log reduction value (LRV) of the virus concentration (refer to, for example, Non-Patent Document 1). For example, the viral clearance capacity in a virus removal step is evaluated by mixing a biological material-containing solution with a virus solution and measuring the respective virus concentrations of the mixture before and after the mixture is brought into contact with a virus removal medium.

With regard to the kind of a virus to be used in evaluating a viral clearance capacity, a guideline is described in q5a of ICT (International Conference on Harmonization of Technical Requirements for Human Use in Europe, the United States, and Japan) and “animal parvoviruses” are given as an example of a useful and non-specific model virus (refer to, for example, Non-Patent Document 1). Among them, minute virus of mice (MVM) and porcine parvovirus (PPV) are frequently used in many viral clearance tests which have quoted the ICH-q5a.

For measuring the virus concentration, mainly used are an infectivity titer measurement method including an endpoint assay method and a local lesion calculation assay method and a quantitative PCR (Quantitative-Polymerase Chain Reaction: qPCR) method (refer to, for example, Non-Patent Documents 2 and 3)

### CITATION LIST

### Patent Documents

Patent Document 1: WO2014/080676A1
Patent Document 2: US Patent Application Publication No. 2012/0088228
Patent Document 3: Japanese Patent No. 4024041

### Non-Patent Documents

Non-Patent Document 1: Virul Safety Evalation of Biotechnology Product Derived from Celline of Human or Animal Origin Q5A (R1)
Non-Patent Document 2: Note For Guidance On Virus Validation Studies: The Design, Contribution And Interpretation Of Studies Validating The Inactivation and Removal Of Viruses
Non-Patent Document 3: Guideline for safety guarantee of plasma fractionated product against virus (Report Number 1047 of the Pharmaceutical and Food Safety Bureau, on Aug. 30, 1999, the notification of the director of the Pharmaceutical and Medical Safety Bureau)
Non-Patent Document 4: Raphael Wolfisberg et al., Journal of Virology (2016)
Non-Patent Document 5: Beatriz Maroto et al., JOURNAL OF VIROLOGY (2004)
Non-Patent Document 6: Ed. by Students' Association/Japanese National Institute of Health, Virus Experiments, particulars: pp. 22-23
Non-Patent Document 7: V. Hutornojs at. al (2012) Env, Exp. Biol. 10: pp. 117-123
Non-Patent Document 8: Anthony M. D'Abramo Jr. et al., 2005 Virology
Non-Patent Document 9: Joshua C Grieger et al., Molecular Therapy 2015
Non-Patent Document 10: Pavel Plevka et al., JOURNAL OF VIROLOGY (2011)
Non-Patent Document 11: PETER TATTERSALL et al., JOURNAL OF VIROLOGY (1976)

### SUMMARY OF INVENTION

### Technical Problem

The present inventors have thought that a viral clearance test method applicable to a continuous process is useful. One of the subjects of the present invention is therefore to provide a viral clearance test method applicable to a continuous process.

### Solution to Problem

[1] A viral clearance test method, including (a) supplying a protein solution to a first channel provided with, upstream and downstream thereof, a protein purification unit and a virus removal filter, respectively, and pouring the protein solution into the protein purification unit at a first constant rate; (b) supplying a virus solution, at a second constant rate, to a second channel connected between the protein purification unit and the virus removal filter in the first channel and mixing the purified protein solution with the virus solution in the first channel; (c) pouring a mixture of the protein solution and the virus solution into the virus removal filter at a third constant rate; and (d) measuring a virus contained in a permeate of the mixture which has passed through the virus removal filter.

[2] The method according to [1], wherein the first channel is provided with a first pump for pouring the protein solution into the protein purification unit at the first constant rate.

[3] The method according to [1] or [2], wherein the second channel is provided with a second pump for pouring the virus solution at the second constant rate.

[4] The method according to any of [1] to [3], wherein the protein solution is continuously poured into the protein purification unit.

[5] The method according to any of [1] to [4], wherein the virus solution is continuously poured into the second channel.

[6] The method according to any of [1] to [5], wherein the mixture is continuously poured into the virus removal filter.

[7] The method according to any of [1] to [6], wherein supposing that a represents the first constant rate, b represents the second constant rate, x represents a virus concentration in the mixture, and y represents a virus concentration in the virus solution, a, b, x, and y satisfy the following formula (1):

x/y=b/(a+b) (1).

[8] The method according to any of [1] to [7], wherein a ratio of the second constant rate to a sum of the first constant rate and the second constant rate is 0.1% or more and 20% or less.

[9] The method according to any of [1] to [8], wherein a virus infectivity titer (Log₁₀ TCID₅₀ (unit/mL)) in the mixture is 2 or more and 10 or less.

[10] The method according to any of [1] to [9], wherein the virus removal filter has a membrane area of 0.0001 m² or more and 4 m² or less.

[11] The method according to any of [1] to [10], wherein a flux of the permeate in the virus removal filter is 0.1 LMH or more and 500 LMH or less.

[12] The method according to [7], wherein supposing that C (m²) represents a membrane area of the virus removal filter, Dₘᵢₙ represents a minimum value of b/(a+b), Dₘₐₓ represents a maximum value of b/(a+b), Fₘᵢₙ (LMH) represents a minimum flux of the permeate in the virus removal filter, and Fₘₐₓ (LMH) represents a maximum flux of the permeate in the virus removal filter,

a minimum value aₘᵢₙ (mL/min) of the first constant rate a is given by the following formula (2):

aₘᵢₙ=(1−Dₘₐₓ)(1000/60)×Fₘᵢₙ×C (2),

a maximum value aₘₐₓ (mL/min) of the first constant rate a is given by the following formula (3):

aₘₐₓ=(1−Dₘᵢₙ)(1000/60)×Fₘₐₓ×C (3),

a minimum value bₘᵢₙ (mL/min) of the second constant rate b is given by the following formula (4):

bₘᵢₙ=Dₘᵢₙ(1000/60)×Fₘᵢₙ×C (4), and

a maximum value bₘₐₓ (mL/min) of the second constant rate b is given by the following formula (5):

bₘₐₓ=Dₘₐₓ(1000/60)×Fₘₐₓ×C (5).

[13] The method according to any of [1] to [12], further including: after pausing the supply of the protein solution into the first channel, supplying the first channel with a washing liquid and pouring the washing liquid into the protein purification unit and the virus removal filter; and measuring a virus contained in a permeate of the washing liquid which has passed through the virus removal filter.

[14] The method according to [13], wherein time until the first channel is supplied with the washing liquid after the supply of the protein solution into the first channel is paused is 0 minutes or more and 24 hours or less.

[15] The method according to [1] to [14], wherein the virus solution contains a protein.

[16] The method according to [1] to [15], wherein the virus solution contains a protein the same as the protein contained in the protein solution.

[17] The method according to or [16], wherein a concentration of the protein in the virus solution is equal to a concentration of the protein in the protein solution.

[18] The method according to any of [1] to [17], further including comparing an amount of the virus contained in the mixture before passing through the virus removal filter with the amount of the virus contained in the permeate of the mixture which has passed through the virus removal filter.

[19] The method according to any of [1] to [18], wherein the protein purification unit has a virus removal capacity.

[20] The method according to [19], wherein a log reduction value (LRV) in the protein purification unit 11 is 0 or more and 7 or less.

### Advantageous Effects of Invention

The present invention makes it possible to provide a viral clearance test method applicable to a continuous process.

### BRIEF DESCRIPTION OF DRAWINGS

FIG. 1 is a schematic view of the protein purification system of an embodiment.

FIG. 2 is a table showing the purification conditions of Examples 1 to 6.

FIG. 3 is a table showing the purification results of Examples 1 to 6.

FIG. 4 is a schematic view of the protein purification system of Comparative Example 1.

FIG. 5 is a table showing the purification conditions of Comparative Examples 1 to 3.

FIG. 6 is a table showing the purification results of Comparative Examples 1 to 3.

FIG. 7 is a schematic view of the protein purification system of Comparative Example 4.

FIG. 8 is a table showing the purification conditions of Comparative Examples 4 to 9.

FIG. 9 is a table showing the purification results of Comparative Examples 4 to 9.

### DESCRIPTION OF EMBODIMENTS

A mode (which may hereinafter be called “embodiment”) for carrying out the present invention will hereinafter be described. The present invention is not limited to the following embodiments and they may be modified in various ways within the range of the gist thereof. The embodiments shown below are only examples of the method for embodying the technical concept of the present invention and the present invention is not limited by these examples.

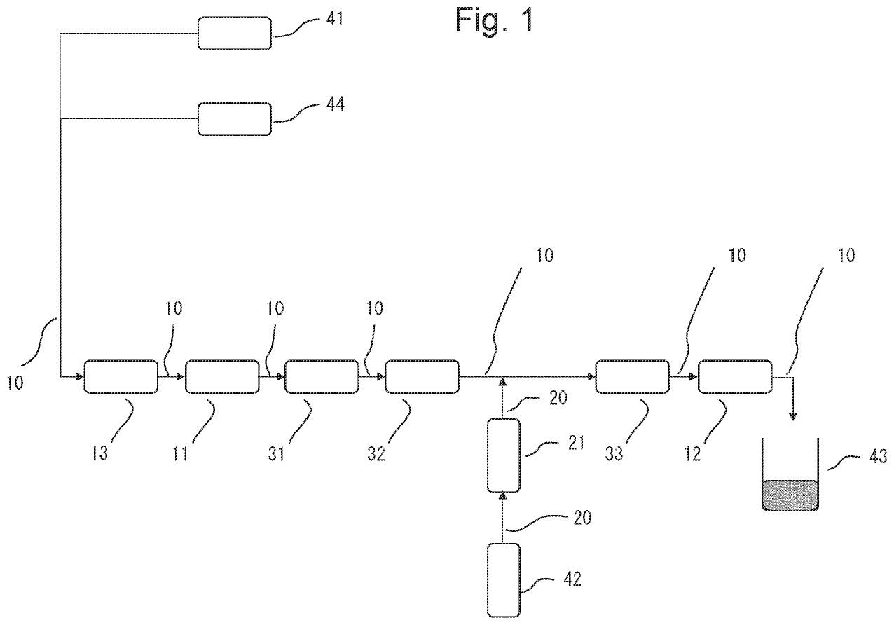

The protein purification system according to the present embodiment has, as shown in FIG. 1, a first channel 10 through which a protein solution flows, a protein purification unit 11 provided upstream of the first channel 10, a virus removal filter 12 provided downstream of the first channel 10, and a second channel 20 connected between the protein purification unit 11 and the virus removal filter 12 in the first channel 10. The first channel 10 may be provided with a first pump 13 for pouring the protein solution into the protein purification unit 11 at a first constant rate. The second channel 20 may be provided with a second pump 21 for pouring a virus solution at a second constant rate.

The method of the viral clearance test method according to the present embodiment is performed using, for example, the protein purification system shown in FIG. 1. The method of the viral clearance test method according to the present embodiment includes (a) supplying a protein solution to a first channel 10 provided with, upstream and downstream thereof, a protein purification unit 11 and a virus removal filter 12, respectively, and pouring the protein solution into the protein purification unit 11 at a first constant rate; (b) supplying a virus solution, at a second constant rate, to a second channel 20 connected between the protein purification unit 11 and the virus removal filter 12 in the first channel 10 and mixing the purified protein solution with the virus solution in the first channel 10; (c) pouring a mixture of the protein solution and the virus solution into the virus removal filter 12 at a third constant rate; and (d) measuring a virus contained in a permeate of the mixture which has passed through the virus removal filter 12.

The protein solution to be poured into the first channel 10 contains a protein. The protein solution preferably does not contain a virus. The protein solution is, for example, poured into the first channel 10 from a protein solution tank 41.

The first pump 13 is provided, for example, upstream of the protein purification unit 11 in the first channel 10 but is not limited thereto. As the first pump 13, a volume pump can be used, but it is not limited thereto. Examples of the volume pump include, but not limited to, a peristaltic pump. The first pump 13 continuously pours the protein solution into the protein purification unit 11 at a first constant rate.

Examples of the protein include antibodies. An antibody is, as generally defined in biochemistry, a glycoprotein molecule (also called “gamma globulin” or “immunoglobulin”) produced by B-lymphocytes as an infection protective mechanism of vertebrate animals. For example, an antibody is used as a pharmaceutical product for humans and may have substantially the same structure as that of an antibody in the human body to which it is administered.

The antibody may be a human antibody or an antibody derived from a nonhuman mammal such as bovine and mouse. Alternatively, the antibody may be a chimeric antibody with human IgG or a humanized antibody. The chimeric antibody with human IgG is an antibody in which variable regions are derived from a nonhuman organism such as mouse and the other constant regions are replaced by human-derived immunoglobulin. The humanized antibody is an antibody having a variable region in which a complementarity-determining region (CDR) is derived from a nonhuman organism and the other framework region (FR) is a human-derived one. The humanized antibody has further reduced immunogenicity than the chimeric antibody.

The class (isotype) and subclass of the antibody are not limited. For example, the antibody is classified according to a difference in the structure of a constant region into five classes such as IgG, IgA, IgM, IgD, and IgE. However, the antibody may belong to any of these five classes. In the human antibody, IgG has four subclasses IgG1 to IgG4 and IgA has two subclasses IgA1 and IgA2. The antibody may belong to any of these subclasses. An antibody-related protein such as Fc fusion protein obtained by binding a protein to a Fc region may also be included in the antibody.

The antibody can be classified according to its origin. However, the antibody may be any of a naturally occurring human antibody, a recombinant human antibody produced by genetic recombination technique, a monoclonal antibody, and a polyclonal antibody. From the standpoint of demand and importance as an antibody drug, human IgG is preferred as an antibody but is not limited thereto.

The protein purification unit 11 removes an impurity contained in the protein and purifies the protein contained in a protein solution. The protein purification unit 11 is equipped with, for example, a chromatography column.

The chromatography column may be, for example, a cation exchange chromatography column. In the cation exchange chromatography column, an aggregate of a protein such as an oligomer in the antibody is adsorbed, as an impurity, to a cation exchange carrier and a protein such as a monomer in the antibody passes through the cation exchange chromatography column.

The cation exchange carrier has a cation exchange group. The cation exchange group may be either a strong cation exchange group or a weak cation exchange group, or it may be both.

The strong cation exchange group generally shows a constant charge amount because it is charged in a pH region of an antibody solution. Accordingly, when the cation exchange carrier has a strong cation exchange group, at least a predetermined charge amount is constantly ensured. When the cation exchange carrier has a strong cation exchange group, a change in charge amount relative to pH is suppressed and improvement in reproducibility of purification properties can be achieved. Examples of the strong cation exchange group include a sulfonic acid group.

The weak cation exchange group can change a charge amount by the pH of a mobile phase. By changing the pH of a mobile phase, therefore, the charge density of the cation exchange carrier can be controlled. This means that by adjusting the pH according to the properties of an impurity to be removed, any intended impurity can be removed. Examples of the weak cation exchange group include a carboxyl group, a phosphonic acid group, and a phosphoric acid group.

Examples of the form of the cation exchange carrier include, but not limited to, membrane, beads, and monolith.

Examples of the cation exchange carrier in the form of membrane include, but not limited to, Mustang (trade name) S (Pall Corporation), Sartobind (trademark) S (Sartorius Stedim Biotech), and Natrix HD-Sb and Natrix HD-C(each, Natrix Separations).

Examples of the cation exchange carrier in the form of beads include, but not limited to, SP Sepharose (trade name) Fast Flow, High Performance, and XL; Capto (trade name) S (GE Healthcare), Fractogel (trademark)COO—, SO₃—, and SE Highcap; Eshumuno (trademark) S and CPX (Merck Millipore Corporation); POROS (trademark) XS and HS (ThermoFisher), Nuvia (trade name) S and HR-S; UNOsphere (trade name) S and Rapid S; Macro-Prep (trademark) High S, CM, and 25 S (Bio-Rad); and Cellufine (trademark) Max CM and Max S (JNC); and Cellufine (trademark) DexS-HbP (JNC).

Examples of the cation exchange carrier in the form of monolith include, but not limited to, CIM (trademark) SO₃ (BIA Separations).

The chromatography column may be, for example, an anion exchange chromatography column. In the anion exchange chromatography column, an impurity having a low isoelectric point such as host cell-derived protein (HCP), nucleic acid, or virus adsorbs to an anion exchange carrier and a protein such as a monomer in an antibody passes through the anion exchange chromatography column.

The anion exchange carrier has an anion exchange group. The anion exchange group may be either a strong anion exchange group or a weak anion exchange group, or it may be both.

Examples of the strong anion exchange group include quaternary ammonium having a trimethylamino group, a triethylamino group, or the like.

Examples of the weak anion exchange group include, but not limited to, a tertiary amine. A tertiary amine having two or more alkyl groups having two or more carbon atoms may have adequate hydrophobicity. Examples of the tertiary amine include a diethylamino group, a dipropylamino group, a diisopropylamino group, and a dibutylamino group.

Examples of the shape of the cation exchange carrier include, but not limited to, membrane, beads, and monolith.

Examples of the anion exchange carrier in the form of membrane include, but not limited to, Chromasorb (trade name) (Merck Millipore Corporation), Mustang (trademark) Q (Pall Corporation), Sarotibind (trademark) Q, STIC (trademark) PA (Sartorius Stedim Biotech), NatriFlo (trademark) HD-Q (Natrix Separations), and QyuSpeed (trade name) D (Asahi Kasei Medical).

Examples of the anion exchange carrier in the form of beads include, but not limited to, Q Sepharose (trade name) Fast Flow, High Performance, and XL; QAE Sephadex (trade name) (GE Healthcare); Fractogel (trade mark) TMAE, TMAE Highcap, DMAE, and DEAE; Eshmuno (trade mark) Q (Merck Millipore Corporation); POROS (trademark) XQ, HQ, D, and PI (ThermoFisher); DEAE-Cellulose (Sigma-Aldrich); Nuvia (trade name) Q; UNOsphere (trade name) Q; Macro-Prep (trademark) High Q, DEAE, and 25 Q (Bio-Rad); CaptoQ (GE Health Care Japan); and Cellufine (trademark) Max DEAE and Max Q (JNC).

Examples of the anion exchange carrier in the form of monolith include, but not limited to, CIM (trademark) QA, DEAE, and EDA (BIA Separations).

The chromatography column may be, for example, a mixed mode chromatography column. In the mixed mode chromatography column, reverse phase chromatography and ion exchange chromatography are used in combination to purify the protein solution. Examples of a carrier used in the mixed mode chromatography include Cellufine MAX IB (JNC).

The protein purification unit 11 may have a virus removing capacity or may not have a virus removing capacity. A log reduction value (LRV) in the protein purification unit 11 may be, for example, 0 or more and 7 or less. The lower limit of LRV in the protein purification unit 11 may be 0 or more, 1 or more, 2 or more, 3 or more, 4 or more, or 5 or more. The upper limit of LRV in the protein purification unit 11 may be 7 or less or 6 or less.

The protein purification unit 11 in the first channel 10 may be provided with, downstream thereof, a protein concentration measuring instrument 31 for measuring the protein concentration of the protein solution which has passed through the protein purification unit 11. The protein concentration measuring instrument 31 measures the protein concentration of the protein solution which has passed the protein purification unit 11, for example, by the ultraviolet absorption method.

The protein purification unit 11 in the first channel 10 may be provided with, downstream of the unit, a conductivity measuring instrument 32 for measuring the conductivity of the protein solution which has passed through the protein purification unit 11. The conductivity measuring instrument 32 measures the conductivity of the protein solution which has passed through the protein purification unit 11, for example, by the AC bipolar method or electromagnetic induction method.

The protein purification unit 11 in the first channel 10 may also be provided with, downstream of the unit, a pH meter for measuring the pH of the protein solution which has passed through the protein purification unit 11, a thermometer for measuring the temperature, and a pressure gauge for measuring the pressure.

The virus solution to be poured in the second channel 20 contains a virus. The virus solution is, for example, poured into the second channel 20 from a virus solution tank 42.

A volume pump is usable as the second pump 21 but the second pump is not limited to it. Examples of the volume pump include, but not limited to a peristaltic pump. The second pump 21 continuously pours the virus solution into the second channel 20 at a second constant rate. The second channel 20 is connected to the first channel 10 so that the protein solution and the virus solution are mixed in the first channel 10 downstream from a connecting point of the second channel 20 and the first channel 10. An in-line mixer 33 may be provided in the first channel 10 downstream from the connecting point of the second channel 20 and the first channel 10. The in-line mixer 33 accelerates the mixing of the protein solution and the virus solution.

The virus may be an infectious virus. The virus may be a naturally-occurring virus. The naturally-occurring virus includes a virus obtained by culturing, in a medium, a host cell infected with a virus and a virus obtained by transfecting a virus nucleic acid into a cell and culturing the resulting cell.

Examples of the virus include, but not limited to, minute virus of mice (MVM), porcine parvovirus (PPV), reovirus type 3, acute poliomyelitis virus (PolioVirus), porcine herpes virus (Pseudorabies Virus), herpes simplex virus type 1 (Human Herpes Virus 1), xenotropic murine leukemia virus (X-MuLV), and bovine viral diarrhea virus.

For example, the virus solution to be supplied to the second channel 20 contains a protein the same as that contained in the protein solution to be supplied to the first channel 10. For example, the concentration of the protein in the virus solution to be supplied in the second channel 20 is equal to that of the protein in the protein solution to be supplied in the first channel 10. The concentration of the protein in the mixture of the protein solution and the virus solution therefore becomes equal to the concentration of the protein in the protein solution supplied in the first channel 10.

The infectivity titer (Log₁₀ TCID₅₀ (unit/mL)) of the virus in the mixture of the protein solution and the virus solution to be poured into the virus removal filter 12 is, for example, 2 or more, 3 or more, or 4 or more. The infectivity titer (Log₁₀ TCID₅₀ (unit/mL)) of the virus in the mixture is, for example, 10 or less, 9 or less, 8 or less, or 7 or less.

Supposing that a represents the first constant rate, b represents the second constant rate, x represents a virus concentration of the mixture, and y represents a virus concentration in the virus solution, the first constant rate, the second constant rate, the supply amount of the protein solution, the supply amount of the virus solution, and the virus concentration in the virus solution may be adjusted so that a, b, x, and y satisfy the following formula (6):

x/y=b/(a+b) (6)

A ratio of the second constant rate to the sum of the first constant rate and the second constant rate is, for example, 0.1% or more, 0.5% or more, 1.0% or more, 1.5% or more, 2.0% or more, or 3.0% or more. A ratio of the second constant rate to the sum of the first constant rate and the second constant rate is, for example, 20% or less, 15% or less, 10% or less, 9% or less, 8% or less, or 7% or less. By adjusting the ratio of the second constant rate to the sum of the first constant rate and the second constant rate to fall within the aforesaid range, the flow of the protein solution is less likely to be influenced largely by the flow of the virus solution.

The mixture of the protein solution and the virus solution continuously flows into the virus removal filter 12 at a third constant rate.

The membrane area of the virus removal filter 12 is, for example, 0.0001 m² or more, 0.0002 m² or more, 0.0003 m² or more, 0.0006 m² or more, 0.0009 m² or more, or 0.0015 m² or more. The membrane area of the virus removal filter 12 is, for example, 4 m² or less, 3 m² or less, 2 m² or less, or 1 m² or less.

The virus removal filter 12 may be in the form of a hollow yarn or a flat membrane.

Examples of the virus removal filter in the form of a hollow yarn include Planova 15N, 20N, and 35N, and BioEx (each, product of Asahi Kasei Medical).

Examples of the virus removal filter in the flat membrane form include Viresolve Pro (EMD Millipore Corporation); Ultipor VF Grade DV20 and DV50 and Pegasus (trade name) SV4 and Grade LV6 (Pall Corporation); Virosart CPV, HC, and HF (Sartorius Stedim Biotech), and NFP (Merck Millipore Corporation).

The flux of the permeate in the virus removal filter 12 is, for example, 0.1 LMH or more, 1.0 LMH or more, 2.0 LMH or more, 4.0 LMH or more, or 10.0 MHL or more. The flux of the permeate in the virus removal filter 12 is, for example, 500 LMH or less, 400 LMH or less, 300 LMH or less, 200 LMH or less, or 100 LMH or less. The flux of the permeate in the virus removal filter 12 is adjusted by the first pump 13 and the second pump 21.

Supposing that C (m²) represents a membrane area of the virus removal filter 12, a represents the first constant rate, b represents the second constant rate, Dₘᵢₙ represents a minimum value of b/(a+b), Dₘₐₓ represents a maximum value of b/(a+b), Fₘᵢₙ (LMH) represents a minimum flux of the permeate in the virus removal filter, and Fₘₐₓ (LMH) represents a maximum flux of the permeate in the virus removal filter, a minimum value aₘᵢₙ (mL/min) of the first constant rate a is given, for example, by the following formula (7):

aₘᵢₙ=(1−Dₘₐₓ)(1000/60)×Fₘᵢₙ×C (7),

A maximum value aₘₐₓ (mL/min) of the first constant rate a is given, for example, by the following formula (8):

aₘₐₓ=(1-Dₘᵢₙ)(1000/60)×Fₘₐₓ×C (8),

A minimum value bₘᵢₙ (mL/min) of the second constant rate b is given, for example, by the following formula (9):

bₘᵢₙ=Dₘᵢₙ(1000/60)×Fₘᵢₙ×C (9).

A maximum value bₘₐₓ (mL/min) of the second constant rate b is given, for example, by the following formula (10):

bₘₐₓ=Dₘₐₓ(1000/60)×Fₘₐₓ×C (10).

The protein solution supplied in the first channel 10 is continuously poured into the first channel 10 provided with the protein purification unit 11 and the virus removal filter 12. The virus solution supplied in the second channel 20 is continuously poured into the second channel 20 and the first channel 10 provided with the virus removal filter 12. The term “continuously poured” means that the solution is poured without pooling the solution in the middle of the channel.

The permeate of the mixture which has passed the virus removal filter 12 is collected, for example, in a permeate collecting container 43. Examples of a method of measuring the virus contained in the permeate of the mixture which has passed through the virus removal filter 12 include, but not limited to, an infectivity titer measurement method and a quantitative PCR method.

The infectivity titer in the infectivity titer measurement method is a unit expressing the concentration of a virus having infectivity. The infectivity titer measurement method includes an end-point assay which determines the minimum infection unit and a count assay of local lesions formed by a virus. As the end-point assay, commonly used is a 50% infection endpoint (TCID₅₀: Tissue culture infectious dose 50) method for determining a dilution ratio at which 50% of the cells are infection positive. It is determined by carrying out stepwise dilution of a virus, inoculating it on at least a predetermined number of culture cells, culturing them for a predetermined term, and judging infection positive/negative. As the local lesion count assay, commonly used is a plaque assay, which determines plaques formed when inoculating a virus to cells cultured in sheet form, overlaying an agar-containing medium to cover the cells, and measuring the number of plaques formed corresponding to the number of inoculated viruses. The infectivity titer is expressed by TCID₅₀ when the TCID₅₀ method is used and by pfu when the plaque assay is used. The pfu is an abbreviation of a plaque forming unit. The infectivity titer per mL is expressed by a unit TCID₅₀/mL or pfu/mL.

By the quantitative PCR, a nucleic acid enclosed in a virus is quantitatively determined. Typically, one virus particle encloses one nucleic acid molecule in a capsid so that the number of nucleic acid molecules becomes equal to the number of virus particles.

Based on the amount of the virus contained in the mixture before passing through the virus removal filter 12 and the amount of the virus contained in the mixture which has passed through the virus removal filter 12, the viral clearance capacity of the virus removal filter 12 is evaluated. The amount of the virus may be expressed by an infectivity titer or the number of particles. The viral clearance capacity of the virus removal filter 12 is evaluated, for example, by a log reduction value (LRV) given by the following formula (11):

LRV=Log₁₀T₁−Log₁₀T₂ (11)

In the above formula, T₁ represents an amount of a virus contained in the mixture before passing through the virus removal filter 12 and T₂ represents an amount of a virus contained in the mixture which has passed through the virus removal filter 12.

When the LRV is larger, the virus removal filter 12 can be evaluated as having a higher viral clearance capacity.

After the supply of the first channel 10 with the protein solution is paused, the first channel 10 may be supplied with a washing liquid. The washing liquid is poured, for example, from a washing liquid tank 44 to the first channel 10. The washing liquid is a solvent containing neither a protein nor a virus. The washing liquid is poured into the protein purification unit 11 and the virus removal filter 12 and the amount of the virus contained in the permeate of the washing liquid which has passed through the virus removal filter 12. The virus removal filter 12 can be evaluated as having a high virus retaining capacity when the amount of the virus contained in the permeate of the washing liquid is small.

The time until the washing liquid is supplied to the first channel 10 after the supply of the first channel 10 with the protein solution is paused is, for example, 0 minutes or more, 5 minutes or more, 10 minutes or more, or 30 minutes or more. The time (process pause) until the washing liquid is supplied to the first channel 10 after the supply of the first channel 10 with the protein solution is paused is, for example, 24 hours or less, 20 hours or less, 10 hours or less, 5 hours or less, or 1 hour or less.

The method of the viral clearance test method according to the present embodiment makes it possible to carry out a viral clearance test with high reproducibility because virus loading to the virus removal filter 12 can be conducted under uniform conditions by supplying the second channel 20 with the virus solution at a constant rate. When the second channel 20 is supplied with the virus solution not at a constant rate, the reproducibility of the viral clearance test may lower. In addition, when the second channel 20 is supplied with the virus solution not at a constant rate, a change in virus spike amount may make it impossible to calculate the log reduction value (LRV).

### Example 1

A system similar to the protein purification system as shown in FIG. 1 was manufactured. As the protein purification unit 11, a 0.5 mL column packed with a mixed mode chromatography carrier (Cellufine MAX IB, JNC) was used. The Cellufine MAX IB has a ligand obtained by partially modifying a polyamine with a butyl group. As the virus removal filter 12, a Planova BioEx (Asahi Kasei Medical) having a membrane area of 0.0003 m² was used.

A protein solution containing 5 mg/ml IgG was prepared using a solvent of pH 6.5 containing 20 mmol/L tris-acetic acid and 100 mmol/L NaCl. A virus solution containing 10% MVM was prepared by adding MVM to the protein solution.

With the first pump 13, 27 mL of the protein solution was poured into the first channel 10 at a first constant rate of 0.225 mL/min. With the second pump 21, 3 mL of the virus solution was poured into the second channel 20 at a second constant rate of 0.025 mL/min. The percentage of the second constant rate in the sum of the first constant rate and the second constant rate was 10%. As a result, a mixture having a virus infectivity titer (Log₁₀ TCID₅₀ (unit/mL)) of 6.884 was poured into the virus removal filter 12 and the flux of the permeate was 50 LHM. The permeate of the mixture was collected.

After the 30 mL solution was poured into the virus removal filter 12, the first pump 13 and the second pump 21 were paused and the system was allowed to stand for 35 minutes. Then, a solvent of pH 6.5 containing 20 mmol/L tris-acetic acid and 100 mmol/L NaCl used as a washing liquid was poured into the first channel 10 at a constant rate of 0.25 mL/min with the first pump 13. A permeate of the washing liquid which had passed through the protein purification unit 11 and the virus removal filter 12 was collected.

A virus infectivity titer in the collected permeate of the mixture was measured. A log reduction value (LRV) in the virus removal filter 12 as calculated from the virus infectivity titer in the permeate of the mixture and the virus infectivity titer in the mixture before passing through the virus removal filter 12 was 5.56 or more.

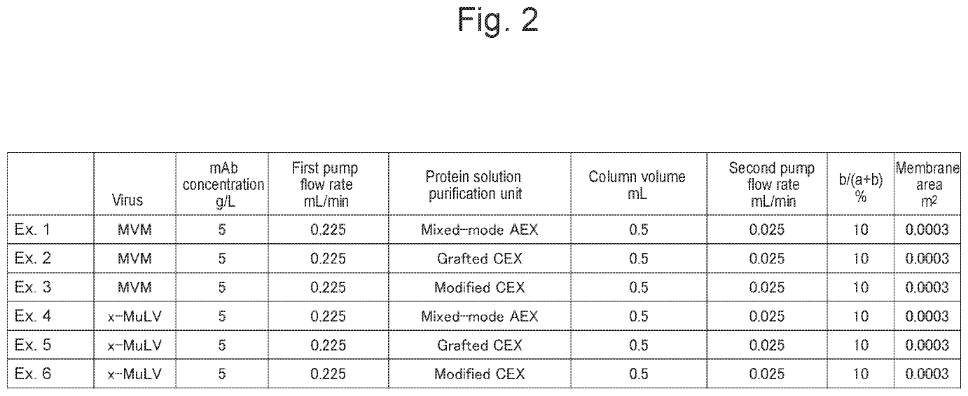

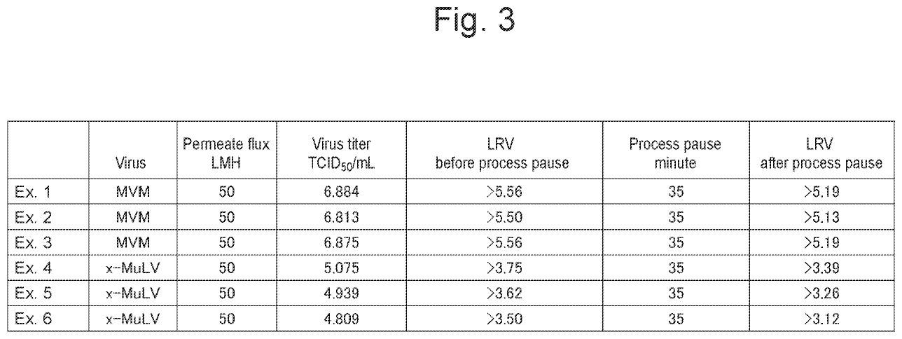

A virus infectivity titer in the collected permeate of the washing liquid was measured. A log reduction value (LRV) in the virus removal filter 12 as calculated from the virus infectivity titer in the permeate of the mixture, the virus infectivity titer in the permeate of the washing liquid, and the virus infectivity titer in the mixture before passing through the virus removal filter 12 was 5.19 or more. The purification conditions and the purification results in Example 1 are shown in FIG. 2 and FIG. 3, respectively.

### Example 2

In a manner similar to that of Example 1 except for the use of a 0.5 mL column packed with a strong cation exchange chromatography carrier (Cellufine MAX GS, JNC) as the protein purification unit 11, the protein solution and the virus solution were poured into the protein purification system. The resulting mixture had a virus infectivity titer (Log₁₀ TCID₅₀ (unit/mL)) of 6.813.

A log reduction value (LRV) in the virus removal filter 12 as calculated from a virus infectivity titer in a permeate of the mixture and the virus infectivity titer in the mixture before passing through the virus removal filter 12 was 5.50 or more.

A log reduction value (LRV) in the virus removal filter 12 as calculated from the virus infectivity titer in the permeate of the mixture, a virus infectivity titer in a permeate of the washing liquid, and the virus infectivity titer in the mixture before passing through the virus removal filter 12 was 5.13 or more. The purification conditions and the purification results in Example 2 are shown in FIG. 2 and FIG. 3, respectively.

### Example 3

In a manner similar to that of Example 1 except for the use of a 0.5 mL column packed with a strong cation exchange chromatography carrier (Cellufine DexS-HbP, JNC) as the protein purification unit 11, the protein solution and the virus solution were poured into the protein purification system. The resulting mixture had a virus infectivity titer (Log₁₀ TCID₅₀ (unit/mL)) of 6.875.

A log reduction value (LRV) in the virus removal filter 12 as calculated from a virus infectivity titer in a permeate of the mixture and the virus infectivity titer in the mixture before passing through the virus removal filter 12 was 5.56 or more.

A log reduction value (LRV) in the virus removal filter 12 as calculated from the virus infectivity titer in the permeate of the mixture, a virus infectivity titer in a permeate of the washing liquid, and the virus infectivity titer in the mixture before passing through the virus removal filter 12 was 5.19 or more. The purification conditions and the purification results in Example 3 are shown in FIG. 2 and FIG. 3, respectively.

### Example 4

In a manner similar to that of Example 1 except for the use of a virus solution containing 10% x-MuLV, the protein solution and the virus solution were poured into the protein purification system. The resulting mixture had a virus infectivity titer (Log₁₀ TCID₅₀ (unit/mL)) of 5.075.

A log reduction value (LRV) in the virus removal filter 12 as calculated from a virus infectivity titer in a permeate of the mixture and the virus infectivity titer in the mixture before passing through the virus removal filter 12 was 3.75 or more.

A log reduction value (LRV) in the virus removal filter 12 as calculated from the virus infectivity titer in the permeate of the mixture, a virus infectivity titer in a permeate of the washing liquid, and the virus infectivity titer in the mixture before passing through the virus removal filter 12 was 3.39 or more. The purification conditions and the purification results in Example 4 are shown in FIG. 2 and FIG. 3, respectively.

### Example 5

In a manner similar to that of Example 2 except for the use of a virus solution containing 10% x-MuLV, the protein solution and the virus solution were poured into the protein purification system. The resulting mixture had a virus infectivity titer (Log₁₀ TCID₅₀ (unit/mL)) of 4.939.

A log reduction value (LRV) in the virus removal filter 12 as calculated from a virus infectivity titer in a permeate of the mixture and the virus infectivity titer in the mixture before passing through the virus removal filter 12 was 3.62 or more.

A log reduction value (LRV) in the virus removal filter 12 as calculated from the virus infectivity titer in the permeate of the mixture, a virus infectivity titer in a permeate of the washing liquid, and the virus infectivity titer in the mixture before passing through the virus removal filter 12 was 3.26 or more. The purification conditions and the purification results in Example 5 are shown in FIG. 2 and FIG. 3, respectively.

### Example 6

In a manner similar to that of Example 3 except for the use of a virus solution containing 10% x-MuLV, the protein solution and the virus solution were poured into the protein purification system. The resulting mixture had a virus infectivity titer (Log₁₀ TCID₅₀ (unit/mL)) of 4.809.

A log reduction value (LRV) in the virus removal filter 12 as calculated from a virus infectivity titer in a permeate of the mixture and the virus infectivity titer in the mixture before passing through the virus removal filter 12 was 3.50 or more.

A log reduction value (LRV) in the virus removal filter 12 as calculated from the virus infectivity titer in the permeate of the mixture, a virus infectivity titer in a permeate of the washing liquid, and the virus infectivity titer in the mixture before passing through the virus removal filter 12 was 3.12 or more. The purification conditions and the purification results in Example 6 are shown in FIG. 2 and FIG. 3, respectively.

### Comparative Example 1

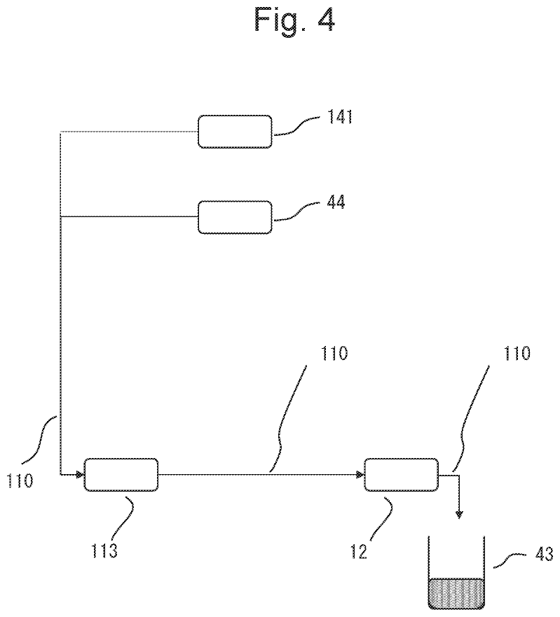

A protein purification system of Comparative Example 1 as shown in FIG. 4 was manufactured. The protein purification system of Comparative Example 1 had a channel 110, a pump 113 provided in the channel 110, and a virus removal filter 12 provided in the channel 110. As the virus removal filter 12, Planova BioEX (Asahi Kasei Medical) having a membrane area of 0.0003 m² was used. The protein purification system of Comparative Example 1 did not have a protein purification unit. In addition, the protein purification system of Comparative Example 1 did not have a second channel and a second pump.

A virus solution containing 1% MVM was prepared using materials similar to those of Example 1. With the pump 113, 30 mL of the virus solution was poured into the channel 110 at a constant rate of 0.025 mL/min, by which the virus solution having a virus infectivity titer (Logo TCID₅₀ (unit/mL)) of 6.13 flowed through the virus removal filter 12 and the flux of the permeate was 5 LHM. The permeate of the virus solution was collected.

After the 30 mL solution flowed through the virus removal filter 12, the pump 113 was paused and the system was allowed to stand for 35 minutes. Then, a washing liquid similar to that of Example 1 was poured into the channel 110 at a constant rate of 0.25 mL/min with the pump 113. A permeate of the washing liquid which had passed the virus removal filter 12 was collected.

A virus infectivity titer in the collected permeate of the virus solution was measured. A log reduction value (LRV) in the virus removal filter 12 as calculated from the virus infectivity titer in the permeate of the virus solution and the virus infectivity titer in the virus solution before passing through the virus removal filter 12 was 5.27 or more.

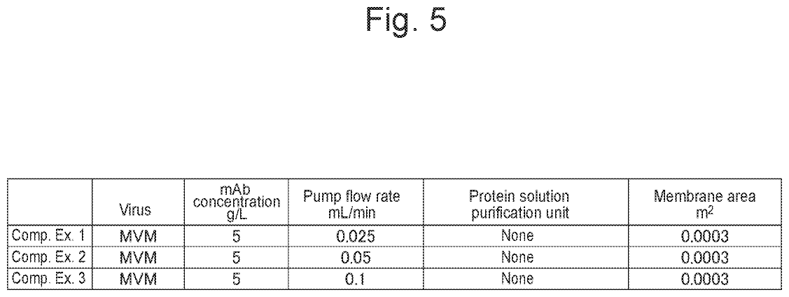

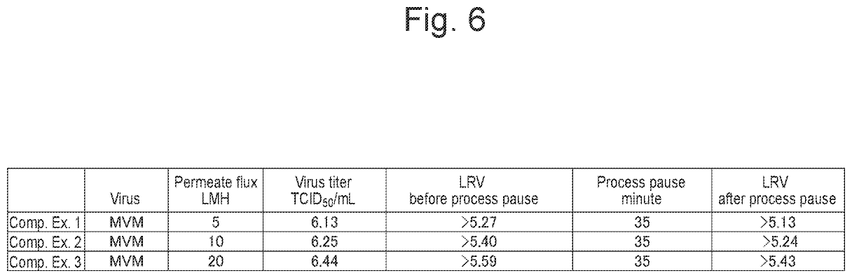

A virus infectivity titer in the collected permeate of the washing liquid was measured. A log reduction value (LRV) in the virus removal filter 12 as calculated from the virus infectivity titer in the permeate of the virus solution, the virus infectivity titer in the permeate of the washing liquid, and the virus infectivity titer in the virus solution before passing through the virus removal filter 12 was 5.13 or more. The purification conditions and the purification results in Comparative Example 1 are shown in FIG. 5 and FIG. 6, respectively.

The LRV obtained in Comparative Example 1 is approximate to the LRV obtained in Examples 1 to 3. This has revealed that in Examples 1 to 3, even when the virus removal filter 12 had, upstream thereof, the protein purification unit 11, the virus removal capacity of the virus removal filter 12 alone was tested.

### Comparative Example 2

In a manner similar to that of Comparative Example 1 except that the virus solution was poured into the channel 110 at a constant rate of 0.05 mL/min, the virus solution was poured into the protein purification system of Comparative Example 1. The virus solution thus poured had a virus infectivity titer (Log₁₀ TCID₅₀ (unit/mL)) of 6.25 and the flux of the permeate was 10 LHM.

A virus infectivity titer in the collected permeate of the virus solution was measured. A log reduction value (LRV) in the virus removal filter 12 as calculated from the virus infectivity titer in the permeate of the virus solution and the virus infectivity titer in the virus solution before passing through the virus removal filter 12 was 5.40 or more.

A virus infectivity titer in the collected permeate of the washing liquid was measured. A log reduction value (LRV) in the virus removal filter 12 as calculated from the virus infectivity titer in the permeate of the virus solution, the virus infectivity titer in the permeate of the washing liquid, and the virus infectivity titer in the virus solution before passing through the virus removal filter 12 was 5.24 or more. The purification conditions and the purification results in Comparative Example 2 are shown in FIG. 5 and FIG. 6, respectively.

### Comparative Example 3

In a manner similar to that of Comparative Example 1 except that the virus solution was poured into the channel 110 at a constant rate of 0.1 mL/min, the virus solution was poured into the protein purification system of Comparative Example 1. The virus solution thus poured had a virus infectivity titer (Log₁₀ TCID₅₀ (unit/mL)) of 6.44 and the flux of the permeate was 20 LHM.

A virus infectivity titer in the collected permeate of the virus solution was measured. A log reduction value (LRV) in the virus removal filter 12 as calculated from the virus infectivity titer in the permeate of the virus solution and the virus infectivity titer in the virus solution before passing through the virus removal filter 12 was 5.59 or more.

A virus infectivity titer in the collected permeate of the washing liquid was measured. A log reduction value (LRV) in the virus removal filter 12 as calculated from the virus infectivity titer in the permeate of the virus solution, the virus infectivity titer in the permeate of the washing liquid, and the virus infectivity titer in the virus solution before passing through the virus removal filter 12 was 5.43 or more. The purification conditions and the purification results in Comparative Example 3 are shown in FIG. 5 and FIG. 6, respectively.

### Comparative Example 4

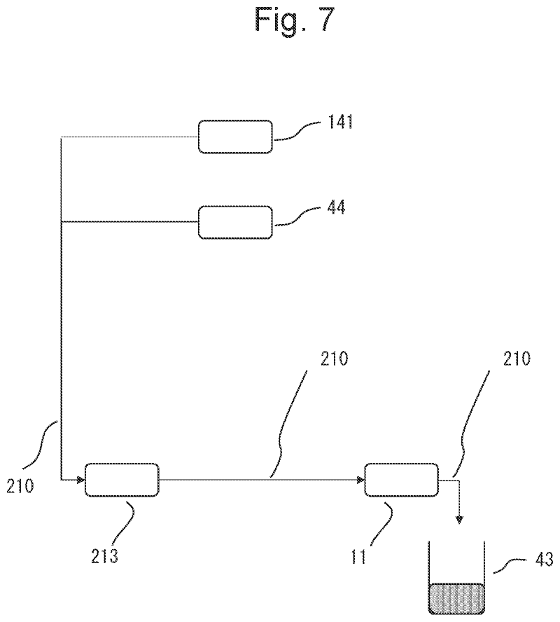

A protein purification system of Comparative Example 4 as shown in FIG. 7 was manufactured. The protein purification system of Comparative Example 4 had a channel 210, a pump 213 provided in the channel 210, and a protein purification unit 11 provided in the channel 210. As the protein purification unit 11, a 0.5 mL column packed with a mixed mode chromatography carrier (Cellufine MAX IB, JNC) was used. The protein purification system of Comparative Example 4 did not have a virus removal filter. In addition, the protein purification system of Comparative Example 4 did not have a second channel and a second pump.

A virus solution containing 5% of MVM was prepared from materials similar to those used in Example 1. The virus solution (30 mL) was poured into the channel 110 at a constant rate of 0.25 mL/min with the pump 213. As a result, the virus solution having a virus infectivity titer (Log₁₀ TCID₅₀ (unit/mL)) of 7.741 flowed through the protein purification unit 11. The permeate of the virus solution was collected.

After 30 mL of the solution flowed through the protein purification unit 11, the pump 213 was paused and the system was allowed to stand for 0.05 minutes. Then, a washing liquid the same as that of Example 1 was poured into the channel 210 at a constant rate of 0.25 mL/min with the pump 213. The permeate of the washing liquid which had passed through the protein purification unit 11 was collected.

A virus infectivity titer in the collected permeate of the virus solution was measured. A log reduction value (LRV) in the protein purification unit 11 as calculated from the virus infectivity titer in the permeate of the virus solution and the virus infectivity titer in the virus solution before passing through the protein purification unit 11 was 3.94.

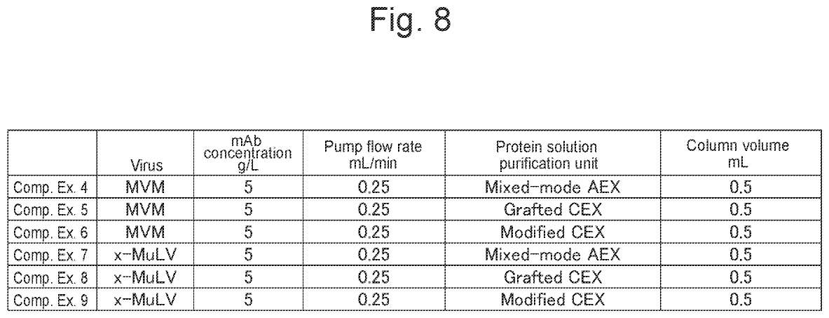

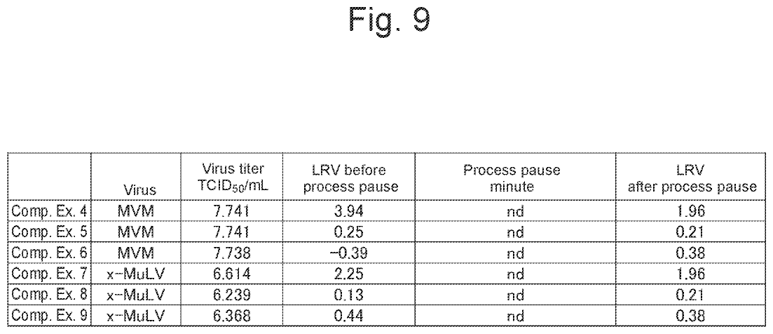

A virus infectivity titer in the collected permeate of the washing liquid was measured. A log reduction value (LRV) in the protein purification unit 11 as calculated from the virus infectivity titer in the permeate of the virus solution, the virus infectivity titer in the permeate of the washing liquid, and the virus infectivity titer in the virus solution before passing through the protein purification unit 11 was 1.96. The purification conditions and the purification results in Comparative Example 4 are shown in FIG. 8 and FIG. 9, respectively.

The above results show that the virus is removed at the protein purification unit 11. The results therefore show that when the virus solution is poured from the upstream of the protein purification unit 11 in the system shown in FIG. 1, the LRV only in the virus removal filter 12 cannot be measured accurately.

### Comparative Example 5

In a manner similar to that of Comparative Example 4 except that a 0.5 mL column packed with a strong cation exchange chromatography carrier (Cellufine MAX GS, JNC) was used as the protein purification unit 11, the virus solution was poured into the protein purification system. The virus solution thus poured having a virus infectivity titer (Log₁₀ TCID₅₀ (unit/mL)) of 7.741 flowed through the protein purification unit 11.

A virus infectivity titer in the collected permeate of the virus solution was measured. A log reduction value (LRV) in the protein purification unit 11 as calculated from the virus infectivity titer in the permeate of the virus solution and the virus infectivity titer in the virus solution before passing through the protein purification unit 11 was 0.25.

A virus infectivity titer in the collected permeate of the washing liquid was measured. A log reduction value (LRV) in the protein purification unit 11 as calculated from the virus infectivity titer in the permeate of the virus solution, the virus infectivity titer in the permeate of the washing liquid, and the virus infectivity titer in the virus solution before passing through the protein purification unit 11 was 0.21. The purification conditions and the purification results in Comparative Example 5 are shown in FIG. 8 and FIG. 9, respectively.

### Comparative Example 6

In a manner similar to that of Comparative Example 4 except that a 0.5 mL column packed with a strong cation exchange chromatography carrier (Cellufine DexS-HbP, JNC) was used as the protein purification unit 11, the virus solution was poured into the protein purification system. The virus solution thus poured having a virus infectivity titer (Log₁₀ TCID₅₀ (unit/mL)) of 7.738 flowed through the protein purification unit 11.

A virus infectivity titer in the collected permeate of the virus solution was measured. A log reduction value (LRV) in the protein purification unit 11 as calculated from the virus infectivity titer in the permeate of the virus solution and the virus infectivity titer in the virus solution before passing through the protein purification unit 11 was −0.39.

A virus infectivity titer in the collected permeate of the washing liquid was measured. A log reduction value (LRV) in the protein purification unit 11 as calculated from the virus infectivity titer in the permeate of the virus solution, the virus infectivity titer in the permeate of the washing liquid, and the virus infectivity titer in the virus solution before passing through the protein purification unit 11 was 0.38. The purification conditions and the purification results in Comparative Example 6 are shown in FIG. 8 and FIG. 9, respectively.

### Comparative Example 7

In a manner similar to that of Comparative Example 4 except for the use of a virus solution containing 10% x-MuLV, the virus solution was poured into the protein purification system of Comparative Example 4. The virus solution thus poured had a virus infectivity titer (Log₁₀ TCID₅₀ (unit/mL)) of 6.614.

A virus infectivity titer in the collected permeate of the virus solution was measured. A log reduction value (LRV) in the protein purification unit 11 as calculated from the virus infectivity titer in the permeate of the virus solution and the virus infectivity titer in the virus solution before passing through the protein purification unit 11 was 2.25.

The virus infectivity titer in the collected permeate of the washing liquid was measured. A log reduction value (LRV) in the protein purification unit 11 as calculated from the virus infectivity titer in the permeate of the virus solution, the virus infectivity titer in the permeate of the washing liquid, and the virus infectivity titer in the virus solution before passing through the protein purification unit 11 was 1.96. The purification conditions and the purification results in Comparative Example 7 are shown in FIG. 8 and FIG. 9, respectively.

### Comparative Example 8

In a manner similar to that of Comparative Example 5 except for the use of a virus solution containing 10% x-MuLV, the virus solution was poured into the protein purification system of Comparative Example 4. The virus solution thus poured had a virus infectivity titer (Log₁₀ TCID₅₀ (unit/mL)) of 6.239.

A virus infectivity titer in the collected permeate of the virus solution was measured. A log reduction value (LRV) in the protein purification unit 11 as calculated from the virus infectivity titer in the permeate of the virus solution and the virus infectivity titer in the virus solution before passing through the protein purification unit 11 was 0.13.

A virus infectivity titer in the collected permeate of the washing liquid was measured. A log reduction value (LRV) in the protein purification unit 11 as calculated from the virus infectivity titer in the permeate of the virus solution, the virus infectivity titer in the permeate of the washing liquid, and the virus infectivity titer in the virus solution before passing through the protein purification unit 11 was 0.21. The purification conditions and the purification results in Comparative Example 8 are shown in FIG. 8 and FIG. 9, respectively.

### Comparative Example 9

In a manner similar to that of Comparative Example 6 except for the use of a virus solution containing 10% x-MuLV, the virus solution was poured into the protein purification system of Comparative Example 4. The virus solution thus poured had a virus infectivity titer (Log₁₀ TCID₅₀ (unit/mL)) of 6.368.

A virus infectivity titer in the collected permeate of the virus solution was measured. A log reduction value (LRV) in the protein purification unit 11 as calculated from the virus infectivity titer in the permeate of the virus solution and the virus infectivity titer in the virus solution before passing through the protein purification unit 11 was 0.44.

A virus infectivity titer in the collected permeate of the washing liquid was measured. A log reduction value (LRV) in the protein purification unit 11 as calculated from the virus infectivity titer in the permeate of the virus solution, the virus infectivity titer in the permeate of the washing liquid, and the virus infectivity titer in the virus solution before passing through the protein purification unit 11 was 0.38. The purification conditions and the purification results in Comparative Example 9 are shown in FIG. 8 and FIG. 9, respectively.

### REFERENCE SIGNS LIST

10: First channel
11: Protein purification unit
12: Virus removal filter
13: First pump
20: Second channel
21: Second pump
31: Protein concentration measuring instrument
32: Conductivity measuring instrument
33: In-line mixer

1. A viral clearance test method, comprising:
supplying a protein solution to a first channel provided with, upstream and downstream thereof, a protein purification unit and a virus removal filter, respectively, and pouring the protein solution into the protein purification unit at a first constant rate;
supplying a virus solution, at a second constant rate, to a second channel connected between the protein purification unit and the virus removal filter in the first channel and mixing the purified protein solution with the virus solution in the first channel;
pouring a mixture of the protein solution and the virus solution into the virus removal filter at a third constant rate; and
measuring a virus contained in a permeate of the mixture which has passed through the virus removal filter.

2. The method according to claim 1, wherein the first channel is provided with a first pump for pouring the protein solution into the protein purification unit at the first constant rate.

3. The method according to claim 1, wherein the second channel is provided with a second pump for pouring the virus solution at the second constant rate.

4. The method according to claim 1, wherein the protein solution is continuously poured into the protein purification unit.

5. The method according to claim 1, wherein the virus solution is continuously poured into the second channel.

6. The method according to claim 1, wherein the mixture is continuously poured into the virus removal filter.

7. The method according to claim 1, wherein supposing that a represents the first constant rate, b represents the second constant rate, x represents a virus concentration in the mixture, and y represents a virus concentration in the virus solution, a, b, x, and y satisfy the following formula (1):
x/y=b/(a+b) (1).

8. The method according to claim 1, wherein a ratio of the second constant rate to a sum of the first constant rate and the second constant rate is 0.1% or more and 20% or less.

9. The method according to claim 1, wherein a virus infectivity titer (Log₁₀ TCID₅₀ (unit/mL)) in the mixture is 2 or more and 10 or less.

10. The method according to claim 1, wherein the virus removal filter has a membrane area of 0.0001 m² or more and 4 m² or less.

11. The method according to claim 1, wherein a flux of the permeate in the virus removal filter is 0.1 LMH or more and 500 LMH or less.

12. The method according to claim 7, wherein supposing that C (m²) represents a membrane area of the virus removal filter, Dₘᵢₙ represents a minimum value of b/(a+b), Dₘₐₓ represents a maximum value of b/(a+b), Fₘᵢₙ (LMH) represents a minimum flux of the permeate in the virus removal filter, and Fₘₐₓ (LMH) represents a maximum flux of the permeate in the virus removal filter,
a minimum value aₘᵢₙ (mL/min) of the first constant rate a is given by the following formula (2):
aₘᵢₙ=(1−Dₘₐₓ)(1000/60)×Fₘᵢₙ×C (2),
a maximum value aₘₐₓ (mL/min) of the first constant rate a is given by the following formula (3):
aₘₐₓ=(1−Dₘᵢₙ)(1000/60)×Fₘₐₓ×C (3),
a minimum value bₘᵢₙ (mL/min) of the second constant rate b is given by the following formula (4):
bₘᵢₙ=Dₘᵢₙ(1000/60)×Fₘᵢₙ×C (4), and
a maximum value bₘₐₓ (mL/min) of the second constant rate b is given by the following formula (5):
bₘₐₓ=Dₘₐₓ(1000/60)×Fₘₐₓ×C (5).

13. The method according to claim 1, further comprising:
after pausing the supply of the protein solution into the first channel, supplying the first channel with a washing liquid and pouring the washing liquid into the protein purification unit and the virus removal filter, and
measuring a virus contained in a permeate of the washing liquid which has passed through the virus removal filter.

14. The method according to claim 13, wherein time until the first channel is supplied with the washing liquid after the supply of the protein solution into the first channel is paused is 0 minutes or more and 24 hours or less.

15. The method according to claim 1, wherein the virus solution contains a protein.

16. The method according to claim 1, wherein the virus solution contains a protein the same as the protein contained in the protein solution.

17. The method according to claim 15, wherein a concentration of the protein in the virus solution is equal to a concentration of the protein in the protein solution.

18. The method according to claim 1, further comprising:
comparing an amount of the virus contained in the mixture before passing through the virus removal filter with the amount of the virus contained in the permeate of the mixture which has passed through the virus removal filter.

19. The method according to claim 1, wherein the protein purification unit has a virus removal capacity.

20. The method according to claim 19, wherein a log reduction value (LRV) in the protein purification unit is 0 or more and 7 or less.
